(19) United States
(12) Patent Application Publication
(10) Pub. No.: US 2009/0264924 A1
(43) Pub. Date: Oct. 22, 2009

(54) Surgical device and method

(76) Inventor: James Ushiba, Monterey, CA (US)

Correspondence Address: PATRICK REILL, P.O. BOX 7218, SANTA CRUZ, CA, 95061-7218, US

(21) Appl. No.: 12/148,519

(22) Filed: Apr. 19, 2008

(51) Int. Cl. A61B 17/04; A61B 19/00

(52) U.S. Cl. 606/228; 128/898

(57) ABSTRACT

A surgical device and method for fixation of a tissue graft into a bone is disclosed. A strand is coupled with the tissue graft. A guide wire is coupled with the strand. The tissue graft is positioned within a socket of the bone and a cannulated screw is driven along the guide wire and into the bone socket to form an interference fixation of the tissue and the tissue graft. The strand attached to the tissue graft is retracted from the bone socket through a strand aperture of the socket. The guide wire is then removed from the bone socket. A loop may be transposed between, and coupling, the tissue graft and the strand. The guide wire may alternatively be coupled with the loop and/or the strand. The tissue graft may be a tendon tissue or a bone-tendon-bone graft.

### FIELD OF THE INVENTION

The present invention relates to surgical devices and methods applied to position organic tissue into a bone structure. More particularly the present invention relates to the need to position a cannulated element in relation to a bone structure.

### BACKGROUND OF THE INVENTION

The prior art provides guide wires that are used to position cannulated elements, e.g., screws, dilators, notchers and taps, for engagement with a human or other mammalian bone. In particular, guide wires are conventionally used to position cannulated screws for the purpose of forming a fixation of a tissue with a bone. The bone may be part of a joint structure, e.g., a human knee. Certain fixation types known in the art that employ interference screws to affix a bone block or a soft tissue graft to a bone include definitive fixation and adjuvant fixation. The prior art also provides for additional methods of fixation, such as endobutton techniques, trans-fixations, and rigid-fixations.

Aperture fixation is a form of adjuvant fixation wherein a bone block or an end of a tendon or other soft tissue length is placed at least partially into a bone socket and an interference screw in driven in between, and engaged with, both a wall of the bone socket and the bone block or soft tissue length. The bone socket is generally surgically formed by drilling a round metallic bit into the bone. The bone socket may be dimensioned in the order of 20 to 35 millimeters in depth and have a cross sectional radius on the order of eight to eleven millimeters.

Some methods of fixation that provide highly stable/strong fixation can be suboptimal if the point of fixation with the bone from which a tendon extends is recessed form or located away from the actual joint or surface of the comprising bone. This distal placement of the point of fixation is not anatomic. A native ligament generally spans the length of a joint and may have one or more additional attachment points within the same joint and a surgical replacement for a native ligament will typically undergo equivalent functional requirements and demands. Furthermore, there is a well-described phenomenon (or “windshield wiper effect”) whereby a reconstructed ligament with a fixation point far from the joint, may move back and forth with joint activity, therefore widening the bone socket aperture, abrading the tendon or other graft material, and causing loosening, failure or degradation of the graft.

Therefore, in an effort to recreate the natural, native anatomy and prevent the windshield wiper effect it is generally preferable to put fixation closer to the joint line, e.g., a surface of the bone from which the surgically affixed tendon extends. In particular, when the screw is used by itself as a definitive fixation element, the screw may be driven into the bone socket so that an exposed end of the screw may be approximately placed up to the level of the bone socket opening, therefore providing aperture fixation.

The prior art further provides for fixating grafts of organic tissue, such as tendon tissue grafts and bone-tendon-bone tissue grafts (hereafter “BTB graft”), into sockets created in bone tissue. Certain prior art methods and devices are directed towards the reconstruction of the anterior cruciate ligament (hereafter “ACL”) using either solely tendon tissue or BTB graft fixation. The prior art generally enables a surgeon to position a tendon tissue graft or a bone element of a BTB graft into a socket drilled into a bone of a patient. In general, prior art methods of ACL reconstruction involve drilling a tunnel through the tibia, drilling a tunnel, i.e., a socket, into the femur, inserting a substitute an ACL tissue graft into the tunnels, and securing the tendon or BTB tissue grafts to the walls of tibial and femoral tunnels and sockets using interference screws.

Although interference screw attachment generally is secure, it is sometimes neither possible nor desirable to provide such fixation, particularly in the femoral socket. In revision situations, for example, where a previous reconstruction has been performed, placing a second femoral tunnel close to the previous socket may not be medically advisable.

A fixation technique that provides strong attachment of a graft in the femoral tunnel using a transverse implant is disclosed in U.S. Pat. No. 5,601,562. The transverse implant is inserted through a loop in a tendon graft. A threaded portion of the implant screws into the bone as the implant is advanced with rotation into the repair site. The technique is disadvantageous, however, because the graft can become wrapped around the implant as it is rotated. In addition, this technique requires a forked insertion tool to lift the tendon graft into the femoral socket, and large tibial and femoral tunnels are needed to accommodate the forked insertion tool. As a result of the large tunnels, the graft can slide laterally and “wipe” back and forth along the fixation implant.

An improved method for loading tendons into a femoral socket is disclosed in U.S. Pat. No. 5,918,604, the entire disclosure of which is incorporated by reference herein. In this technique, a strand of suture or nitinol wire is drawn transversely across the femoral socket, and a loop of the strand is pulled down from the socket and out of the tibial tunnel. The tendon graft is passed through the loop, and the strand loop with tendon attached is lifted back into the femoral socket. A transverse implant is then advanced under the tendon graft, preferably by impact insertion to avoid wrapping of the tendon graft during insertion.

U.S. Pat. No. 7,077,863 (hereafter '863) discloses an improved fixation technique, particularly in cruciate ligament reconstructions, utilizing a BTB graft, or construct, wherein a surgeon passes a strand through an opening of a femoral tunnel and through both the femoral tunnel and a tibial tunnel. The strand is then attached to a loop extending from a bone block of a BTB graft, and the strand is pulled back through the tibial tunnel and into the femoral tunnel, whereby the bone block proximate to the strand is pulled into the femoral tunnel. The surgeon must then insert a guide wire into the femoral tunnel and between the sidewall of the femoral tunnel and the BTB bone block positioned therein. The surgeon may then guide a cannulated implant, such as a fixating screw, along the guide wire and drive the implant to force a friction fit between the bone block, or alternatively a tendon graft, and the sidewall of the femoral tunnel. '863 advises that a nitinol wire may be used as a guide wire, and that the nitinol wire may be introduced after the bone block has been lifted into a femoral tunnel. Accordingly, there is a need for an improved technique to aid the surgeon in positioning the guide wire in concert with the positioning of a tendon or bone element of a tissue graft within a socket of a bone.

### OBJECTS OF THE INVENTION

It is an object of the method of the present invention to provide a method for positioning a guide wire relative to tissue graft and a bone socket.

It is an additional object of certain alternate preferred embodiments of the method of the present invention to provide a device for driving a cannulated element into a bone socket.

It is another additional object of certain still alternate preferred embodiments of the method of the present invention to provide a method for driving a cannulated element between an inserted soft tissue graft, e.g., tendon tissue, and a sidewall of a bone socket to create support a grafting of the inserted soft tissue graft and the bone tissue.

### SUMMARY OF INVENTION

Towards these and other objects that will be made obvious in light of the present disclosure, the method of the present invention provides a guide wire useful in surgical procedures. The guide wire may include a flexible tail length, a more rigid tip length and an attachment feature. Alternatively, the entire length of the guide wire may be substantially uniformly flexible, preferably having a tail and tip as flexible and rugged as a length of nitinol wire having across-section in the range of 0.5 millimeters to 2.0 millimeters.

The guide wire is configured to allow a cannulated screw to travel along the guide for insertion into bone, tendon or other tissue. The guide wire is further configured to enable a withdrawal of the tip length and the attachment feature through an internal channel of the cannulated screw after the cannulated screw has been inserted into bone, tendon or other tissue.

In certain alternate preferred embodiments of the method of the present invention the invented guide wire may be applied to position a dilator, notcher, or tap in relation to a bone or other body part or organ.

The attachment point is configured to enable the guide wire to be attached to a suture and to be positioned by movement of the suture into a proximity of a bone, tendon, or other tissue that the cannulated screw may be engaged with.

In certain alternate preferred embodiments of the present invention the tail length (hereafter, “tail”) may be or comprise a metal wire, a nitinol wire, a surgical suture stitching (hereafter, “suture”), a reinforced suture or thread, or a cord.

Alternatively or additionally, in certain other alternate preferred embodiments of the method of the present invention the tip length (hereafter, “tip”) may be or comprise a wire, a metal wire, nitinol, and/or a biodegradable wire.

Further alternatively or additionally, in certain other alternate preferred embodiments of the method of the present invention a tapered section may be disposed between the tip and the tail in order to reduce a possibility that the cannulated screw will be obstructed when being driven along the tail and onto the tip.

Even further alternatively or additionally, in certain other alternate preferred embodiments of the method of the present invention a the tip and the tail may present an equal or substantially equal radius in order to reduce a possibility that the cannulated screw will be obstructed when being driven along the tail and onto the tip.

In certain yet other alternate preferred embodiments of the method of the present invention the attachment feature may be or comprise a hook, a pair of hooks, an eye, one or more threads, and/or one or more wires.

The foregoing and other objects, features and advantages will be apparent from the following description of the preferred embodiment of the invention as illustrated in the accompanying drawings.

### INCORPORATION BY REFERENCE

U.S. Pat. Nos. 7,329,281; 7,217,280; 7,211,111; 7,083,647; 7,077,863; 6,939,379; 6,833,005; 6,743,233; 6,599,289; 6,221,107; 6,146,408; 5,211,647; and 5,320,626; and US Patent App. Publication Serial Numbers 20060293689; 20060247642; 20060189991; and 20060149259 are incorporated herein by reference and for all purposes. In addition, each and all publications, patents, and patent applications mentioned in this specification are herein incorporated by reference to the same extent in their entirety and for all purposes as if each individual publication, patent, or patent application was specifically and individually indicated to be incorporated by reference.

### BRIEF DESCRIPTION OF DRAWINGS

These, and further features of the invention, may be better understood with reference to the accompanying drawings depicting the preferred embodiment, in which:

FIG. 1A is an illustration of a first version guide wire of the present invention comprising a nitinol guide wire shaped into a tail, a tip, and a strand attachment feature;

FIG. 1B is an illustration of a second version guide wire of the present invention comprising a second guide wire having flexible tail, a nitinol wire tip, and a nitinol wire attachment feature;

FIGS. 2A and 2B are respectively a detailed cut-away a side view and a front view of the first version guide wire of FIG. 1A coupled with a cannulated screw;

FIGS. 3A through 3C illustrate a first version of the method of the present invention, wherein a guide wire is attached to a strand and the guide wire is pulled into a bone socket by the strand as the strand also pulls in a bone block to the same bone socket;

FIG. 4A is a close-up side view of the tip and attachment feature of the first version guide wire of FIG. 1A;

FIG. 4B is a front view of the first version guide wire of FIG. 1A;

FIG. 5A is a side view of a third version guide wire of the present invention having a flexible tail single hook attachment feature;

FIG. 5B is a front view of the third version guide wire of FIG. 5A;

FIG. 6A is a side view of a fourth version guide wire of the present invention having a flexible waxed tail and a double hook attachment feature;

FIG. 6B is a front view of the fourth version guide wire of FIG. 6A;

FIG. 7A is a fifth version guide wire of the present invention having a flexible tail with a Kevlar layer and a double looped hook attachment feature;

FIG. 7B is a front view of the fifth version guide wire of FIG. 7A;

FIG. 8A is a side view of a sixth version guide wire of the present invention having a clamp attachment feature;

FIG. 8B is a front view of the sixth version guide wire of FIG. 8A;

FIG. 9A is a side view of a seventh version guide wire of the present invention having an cord attachment feature;

FIG. 9B is a front view of the seventh version guide wire of FIG. 9A;

FIG. 10 is a second version of the method of the present invention wherein a guide wire of FIG. 1A is attached to a strand of FIG. 1A and the strand is positioned within a fold of flexible material, such as a tendon, and whereby the flexible material may be pulled into a bone socket along with the guide wire; and

FIG. 11 is a side view illustration of a guide wire formed in accordance with the method of the present invention;

FIG. 12 illustrates the method of the bone-tendon-bone graft wherein a guide wire is attached to a strand;

FIG. 13 illustrates an alternate preferred embodiment of the invented guide wire wherein the cross-sectional length of the tip and the tail guide wire are substantially uniform; and

FIG. 14 illustrates an invented tapered guide wire having a tapered section disposed between the tip and a tapering tail.

### DETAILED DESCRIPTION OF A PREFERRED EMBODIMENT

In describing the preferred embodiments, certain terminology will be utilized for the sake of clarity. Such terminology is intended to encompass the recited embodiment, as well as all technical equivalents, which operate in a similar manner for a similar purpose to achieve a similar result.

It is understood that elements depicted in the drawings may be embodied in varying dimensions are that the drawings are therefore not presented in a particular scalar representation but as representative sizes.

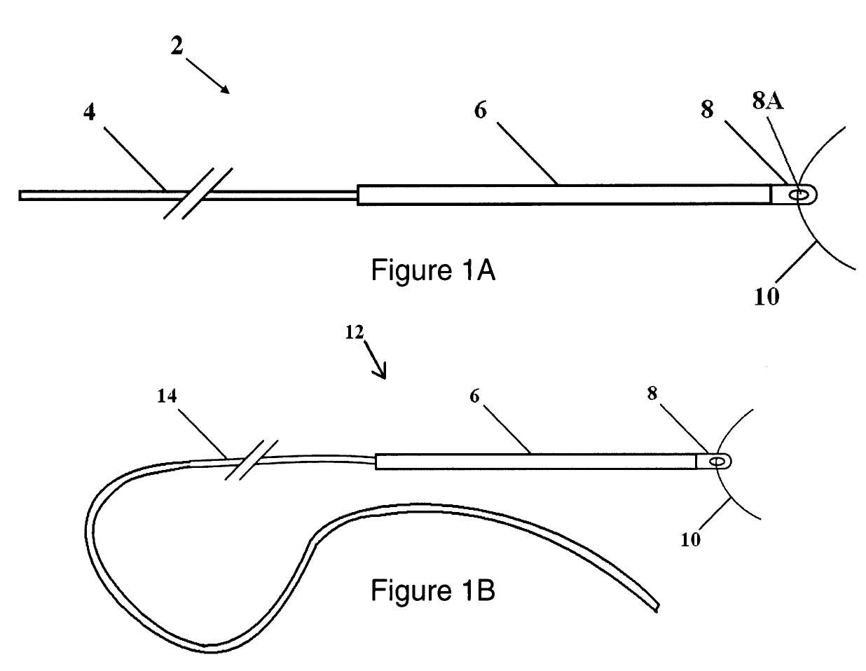

Referring now generally to the Figures and particularly to FIG. 1A, FIG. 1A is an illustration of a first version 2 of the present invention comprising a nitinol guide wire shaped into a tail 4, a tip 6, and a strand attachment feature 8. The strand attachment feature 8, or first attachment feature 8, is configured with an eye 8A that is sized and shaped to allow a surgical suture or wire 10 (hereafter “strand” 10) to extend through the eye 8A. It is understood the strand 10 and the tail 4 are not shown to scale in the Figures in general, so as to make clear the design of the several embodiments of the present invention as disclosed herein.

Referring now generally to the Figures and particularly to FIG. 1B, FIG. 1B is an illustration of a second version guide wire of the present invention 12 comprising a flexible tail 14, the nitinol wire tip 6, and the first attachment feature 8. The flexible tail 14 may comprise or consist of a surgical suture, preferably of number two surgical gauge or number five surgical gauge.

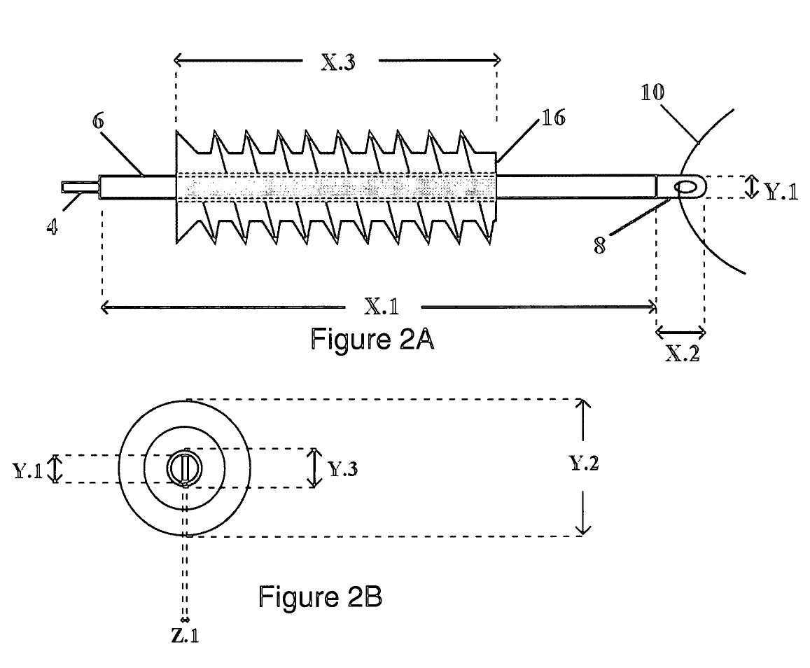

Referring now generally to the Figures and particularly to FIGS. 2A and 2B, FIGS. 2A and 2B are respectively a detailed cut-away a side view and a front view of the first version guide wire 2 of FIG. 1A coupled with a cannulated screw 16. The nitinol wire tip 6 preferably has a tip cross sectional diameter Y.1 of from 1.0 millimeters to 3 millimeters and a linear length X.1. The first attachment feature 8 has a linear length X.2 and preferably a attachment feature maximum cross sectional dimension no greater than the tip cross sectional diameter Y.1 of the nitinol wire tip 6, whereby the first attachment feature 8 and the nitinol wire tip 6 may be withdrawn through a channel 16A of a cannulated screw 16. The cannulated screw 16 has a screw linear length X.3 in the range of from ten millimeters to twenty millimeters.

Referring now to FIG. 2B, FIG. 2B is a front of the cannulated screw 16, the tip 6 and the first attachment feature 8. The maximum outer diameter Y.2 of the cannulated screw 16 is preferably in the range form six millimeters to twelve millimeters. The screw channel 16A presents a channel diameter Y.3, wherein the channel diameter Y.3 is preferably larger than the tip cross sectional diameter Y.1 to enable the cannulated screw 16 to traverse from the tail 4 to and beyond the first attachment feature 8 by passing the first guide wire version 2 through the screw channel 16A. The first attachment feature 8 presents a thickness dimension Z.1 preferably on the order of 0.25 millimeters to 1.5 millimeters, or one fourth to one half of the tip cross sectional diameter Y.1 of the nitinol wire tip 6.

It is understood that the invented guide wire 2 or 12 may also be applied to position a dilator, notcher, or tap in relation to a bone or other body part or organ.

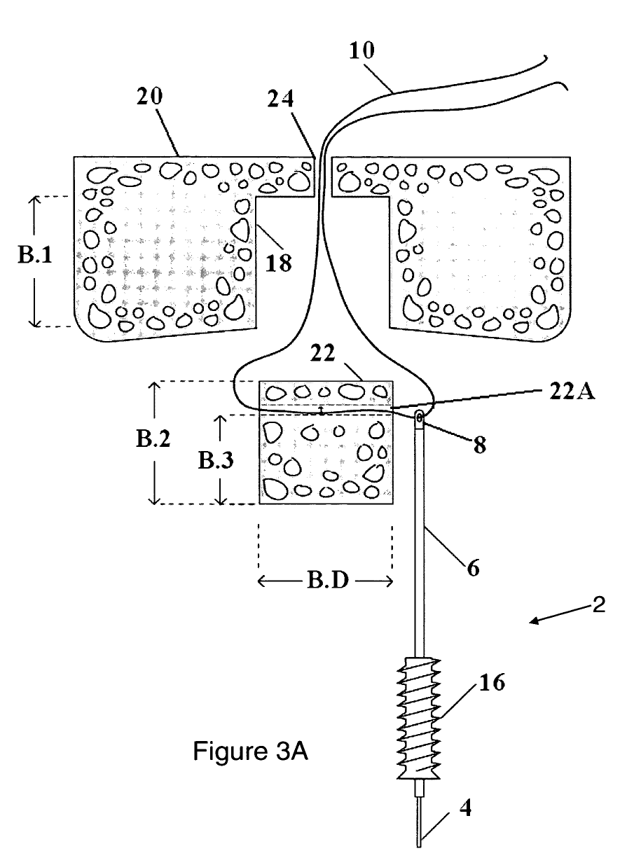

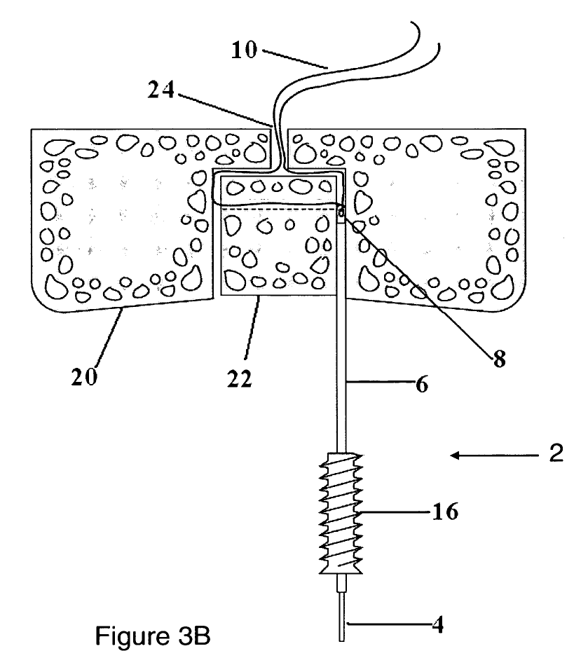

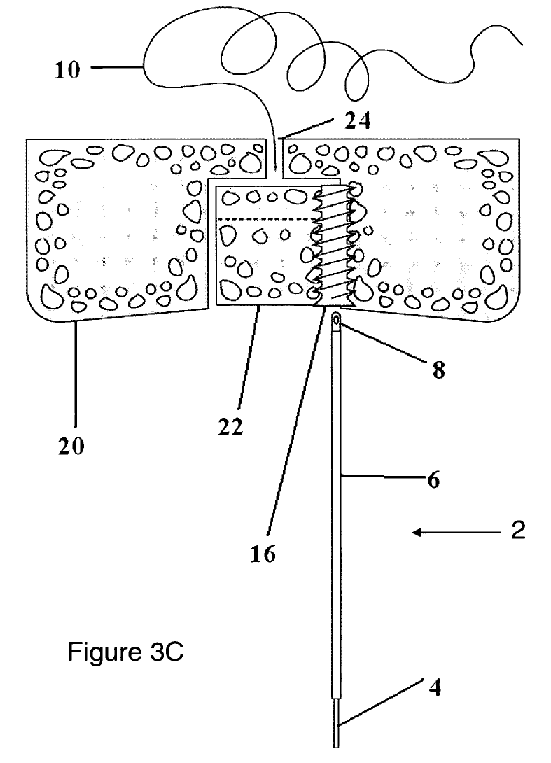

Referring now generally to the Figures and particularly to FIGS. 3A through 3C, FIGS. 3A through 3C illustrate a first version of the method of the present invention, wherein the first guide wire version 2 is attached to the strand 10 and the first version guide wire 2 is pulled into a bone socket 18 of a bone 20 by the strand as the strand 10 also pulls in a bone block 22 into the bone socket 18. It is understood that the bone 20 may be a human bone, such as a femur or a tibia. It is further understood that the preferred embodiment of the method of the present invention is not limited to use of the first guide wire version 2, and may be conducted using any of the invented guide wire configurations disclosed herein. The bone socket 18 has a socket depth B.1 preferably within the range of from ten millimeters to 40 millimeters, and preferably two millimeters to ten millimeters greater than a bone block height B.2. The bone block height B.2 is preferably in the range from ten millimeters to 35 millimeters. The bone block 22 additionally has a bone block channel 22A that is sized to enable the strand 10 to extend there through, and is located at a bone block channel depth B.3 within the bone block 22. The bone block 22 is preferably sized and shaped to enable a friction fit when the bone block 22 is pulled within the bone socket 18, and may be shaped as a cylinder having a bone block height B.2 and a cylindrical diameter B.D within the range of eight millimeters to eleven millimeters.

FIG. 3A illustrates the bone block 22 as positioned prior to a surgeon pulling the bone block 22 into the bone socket 18 by pulling the strand 10 through a bone aperture 24. FIG. 3B illustrates the first version guide wire 2 pulled in between the bone block 22 and the bone socket 18 and positioned to enable a preferred positioning of the cannulated screw 16 for engagement with the bone block 22 and the bone 20. FIG. 3C illustrates the cannulated screw 16 fully engaged with the bone block 22 and the bone 20. FIG. 3C further illustrates the (a.) removal of the strand 10 from the guide wire attachment feature 8 and the bone block 22; and (b.) withdrawal of the tip 6 and the attachment feature 8 through the screw channel 16A and away from the bone 20.

It is understood that a close positioning of a top plane 22B of the bone block 22 in relation to an outer surface 20A of the bone 20 can reduce wear and tear of a tendon of a BTB graft that may be caused by the windshield wiper effect of the tendon rubbing against the bone 20. The method of the present invention enables positioning of the bone block top plane 22B within zero to ten millimeters of the bone top surface 20A, and preferably within three millimeters to five millimeters.

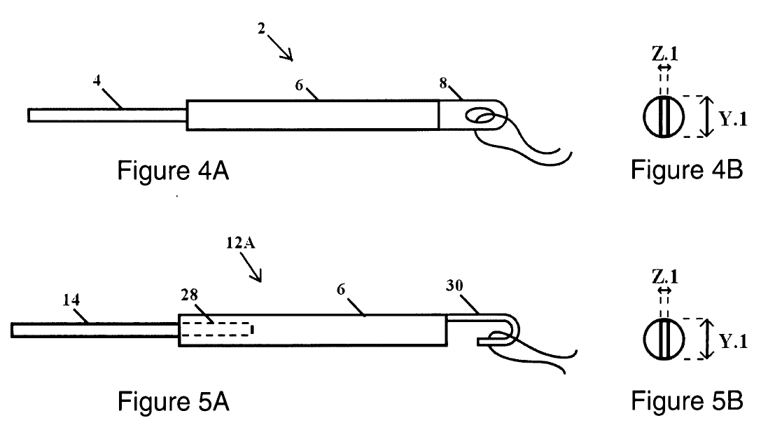

Referring now generally to the Figures and particularly to FIGS. 4A and 4B, FIG. 4A is a close-up side view of a partial length of the nitinol tail 4, the nitinol tip 6 and first version attachment feature 8 of the first version guide wire 2 of FIG. 1A. FIG. 4B is a front view of the first version guide wire 2, wherein the first attachment feature has a maximum width dimension Z.1 and a height of Y.1.

Referring now generally to the Figures and particularly to FIGS. 5A and 5B, FIG. 5A is cut away side view of a third version 12A of the flexible tail guide wire 12 of the present invention having a flexible tail single hook attachment feature 30, or single hook 30. The single hook 30 comprises a nitinol wire extending from the nitinol tip 6. The single hook 30 has a cross-sectional diameter preferably from 0.5 millimeters to one millimeter and is sized and shaped to substantially conform around and capture the strand 10. A flexible tail portion 28 is friction fit within the nitinol tip 6 to ensure that the flexible tail 14, or suture 14, remains coupled with the nitinol tip 6 during the execution of the method of the present invention.

FIG. 5B is front view of the third version 12A of the flexible tail guide wire 14, wherein the single hook 30 is shown to have a single hook diameter Z.1, wherein the single hook diameter Z.1 is preferably in the range of 0.25 to 0.5 of the cross sectional wire diameter Y.1 of the nitinol wire tip 6.

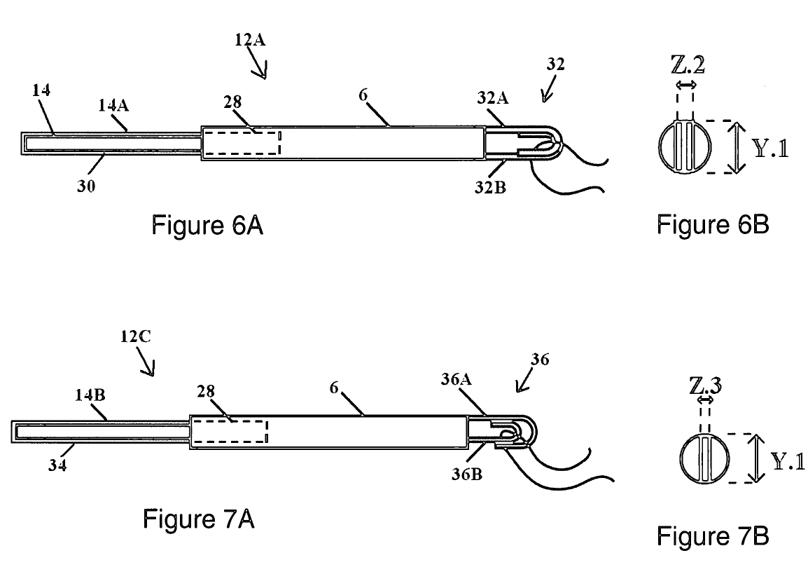

Referring now generally to the Figures and particularly to FIG. 6A, FIG. 6A is a fourth version guide wire 12B of the present invention having a flexible waxed tail 14A and a double hook attachment feature 32, or double hook 32. The flexible waxed tail 14A includes the suture 14 and a wax layer 30, wherein the flexible waxed tail 14A preferably has a cross sectional diameter no greater than the cross sectional diameter Y.1 of the nitinol wire tip 6. The flexible tail portion 28 is friction fit within the nitinol tip 6 to ensure that the flexible waxed tail 14A remains coupled with the nitinol tip 6 during the execution of the method of the present invention. The single hook 30 comprises a nitinol wire extending from the nitinol tip 6. The double hook 32 includes a first hook 32A and a second hook 32B that comprise nitinol wire and each extend from the nitinol tip 6, and each have a cross sectional diameter preferably in the range from 0.125 to 0.5 of the tip cross sectional diameter Y.1. The first hook 32A and the second hook 32B each have a cross-sectional diameter preferably from 0.25 millimeters to 1.0 millimeter and are each sized and shaped to substantially conform around and capture the strand 10.

FIG. 6B is a front view of the fourth version guide wire 12B and illustrates a combined dimension Z.2 of the cross sectional diameters of the first hook 32A and the second hook 32B that is preferably within the range from 0.5 millimeters to 2.0 millimeters, or within the range of one half to equal to the cross sectional diameter Y.1 of the nitinol wire tip 6.

Referring now generally to the Figures and particularly to FIG. 7, FIG. 7 is a fifth version guide wire 12C of the present invention having an alternate flexible tail 14B with a Kevlar layer 34 and a double looped attachment feature 36. The alternate flexible waxed tail 14B includes the suture 14 and a Kevlar layer 34, wherein the alternate flexible waxed tail 14B preferably has a cross sectional diameter no greater than the cross sectional diameter Y.1 of the nitinol wire tip 6. The flexible tail portion 28 is friction fit within the nitinol tip 6 to ensure that the alternate flexible waxed tail 14B remains coupled with the nitinol tip 6 during the execution of the method of the present invention. The Kevlar layer 34 serves to make the alternate waxed tail 14B more rugged than the suture 14 is alone.

The double looped attachment feature 36 includes an outer loop 36A and an inner loop 36B, each loop 36A & 36B comprising nitinol wire having a cross sectional diameter Z.3 preferably within the range for 0.5 millimeters to one millimeter, and/or within the range form one fourth to one half of the diameter Y.1 of the nitinol wire tip 6. Inner loop 36B and the outer loop 36A are sized and shaped to capture the strand 10.

FIG. 7B is a front view of the fifth version guide wire 12C of FIG. 7A wherein the cross sectional diameter Y.1 nitinol guide sire tip 6 and the cross sectional diameter Z.3 of the double looped attachment feature 36 are presented.

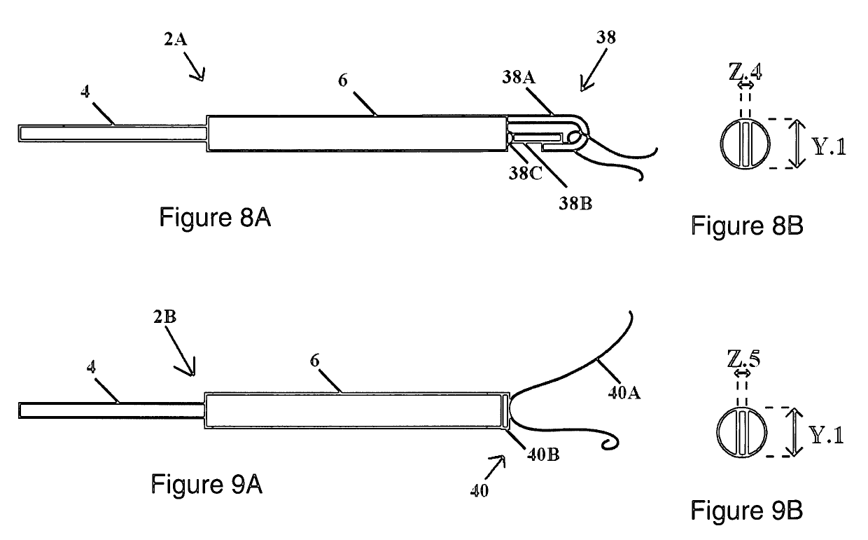

Referring now generally to the Figures and particularly to FIG. 8A, FIG. 8A is a sixth version nitinol guide wire 2A of the present invention having a clamp attachment feature 38, or clamp 38. Clamp 38 includes a clamp hook 38A and a spring biased lever 38B. The spring biased lever 38B presses against, and is captured by, the clamp hook 38A to enable the clamp 38 to retain the suture 10. The clamp hook 38A and the spring biased lever 38B are sized and shaped to surround and secure the strand 10. The spring biased lever 38B includes a lever point 38C that permits rotational motion of the spring biased lever 38B away from the clamp hook 38A when force is applied to open the clamp 38 to either insert or remove the strand 10 from within the clamp 38.

FIG. 8B is a front view of the nitinol wire tip 6 and the clamp 38, wherein the nitinol wire tip is shown to have a cross sectional diameter Y.1 preferably within a range from one millimeter to three millimeters, and the clamp 38 has a cross sectional wire diameter Z.4 in the range from 0.25 to 1.5 millimeters, or from one fourth to one half of the nitinol tip cross sectional wire diameter Y.1.

Referring now generally to the Figures and particularly to FIG. 9A, FIG. 9A is a seventh version guide wire 2B of the present invention having a cord attachment feature 40, or cord assembly 40. The cord assembly 40 includes a surgical suture 40A preferably of number two surgical gauge or number five surgical gauge and preferably having a length in the range from five to fifty millimeters, whereby the surgical suture 40A may be tied or knotted about the strand 10 to couple the seventh version guide wire 2B to the strand 10. An adhesive 40B of the cord assembly 40 attaches the surgical suture 40A to the guide wire tip 6.

FIG. 9B is a front view of the seventh version guide wire 2B wherein the surgical suture 40A is shown to have a suture diameter Z.5 preferably in the range between 0.5 millimeters to 0.7 millimeters.

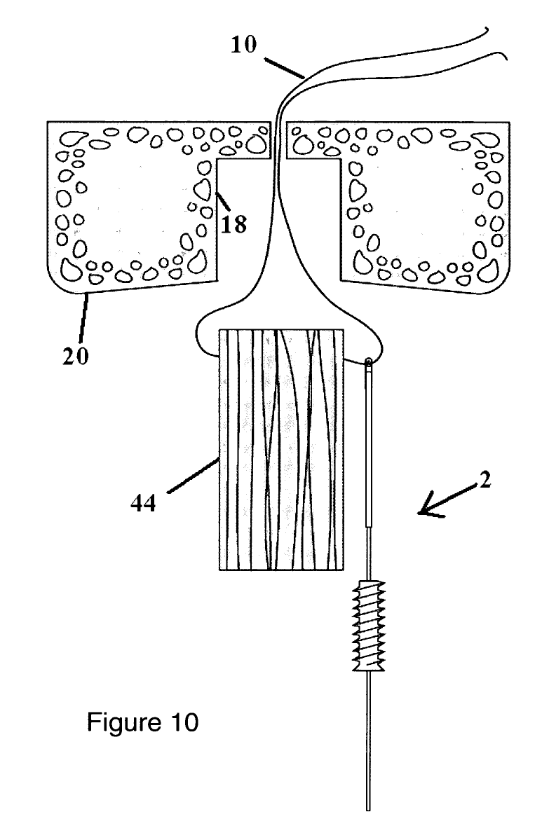

Referring now generally to the Figures and particularly to FIG. 10, FIG. 10 is a second version of the method of the present invention wherein a nitinol guide wire 2 is attached to the strand 10 and the strand 10 is positioned within a fold of flexible material 44, such as a tendon, and whereby the flexible material 44 may be pulled into a bone socket 18 along with the nitinol guide wire 2.

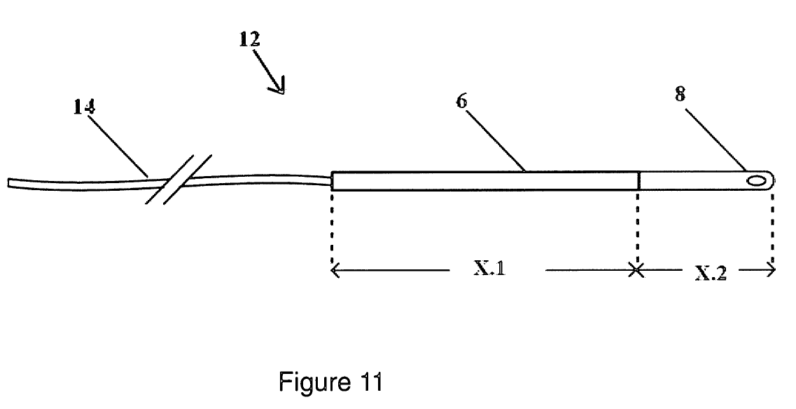

Referring now generally to the Figures and particularly to FIG. 11, FIG. 11 is a side view illustration of a flexible guide wire 12 formed in accordance with the method of the present invention. The tip length X.1 and the attachment feature length X.2 are preferably in combination equal to or greater than the sum of the bone block height B.2 and the screw length X.3.

It is understood that the tip 6 may be formed of nitinol, or other suitable metallic, nonmetallic, plastic and/or biodegradable material known in the art.

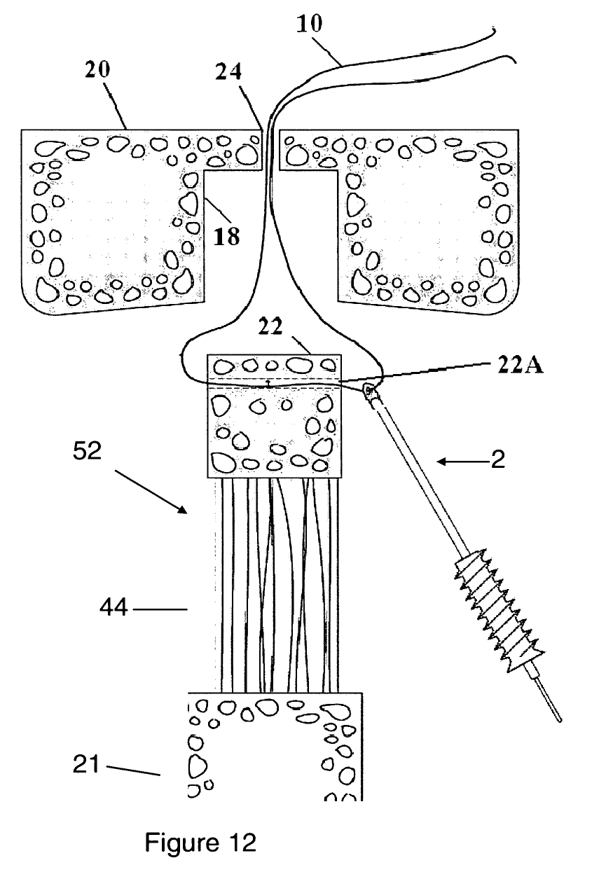

Referring now generally to the Figures and particularly to FIG. 12, FIG. 12 is a third version of the method of present invention using a BTB graft 52 wherein a nitinol guide wire 2 is attached to the strand 10 and the strand 10 is positioned within a bone block channel 22A within a bone block 22 which is affixed to a flexible material 44, such as a tendon, which is then affixed to an independent bone 21, whereby the BTB graft 52 may be pulled into a bone socket 18 along with the nitinol guide wire 2. It is understood that the independent bone 21 may be substituted for another bone block 22.

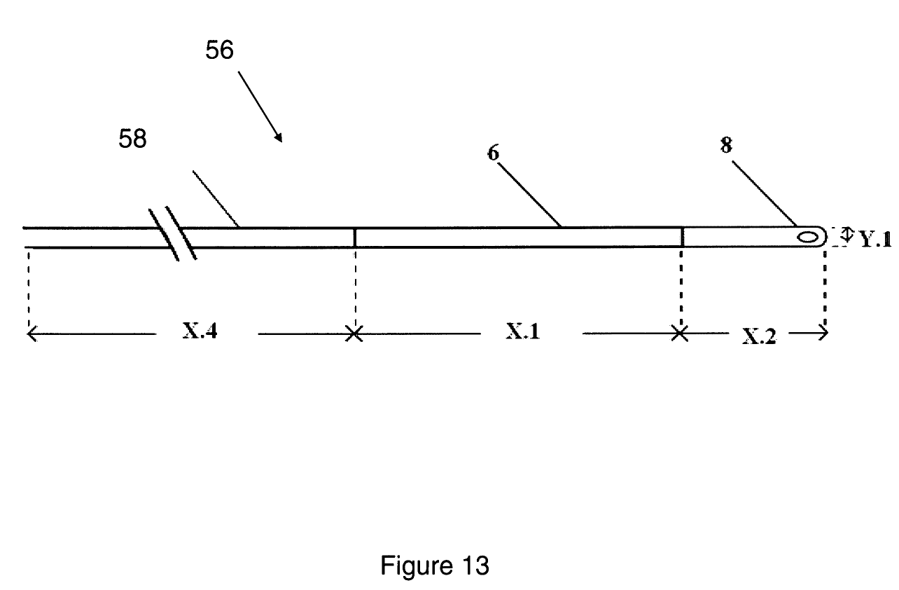

Referring generally to the Figures and particularly to FIG. 13, FIG. 13 illustrates a uniform invented guide 56. The uniform guide wire 56 comprises a uniform tail 58, the tip 6 and the needle attachment feature 8, wherein all three elements 58, 6 & 8 of the uniform guide wire 56 fit with a form factor of a flexible wire having a uniform radius Y.1. For example, the uniform tail 58 may be made of nitinol wire or a suture have a radius Y.1 that equal to, or substantially equal to, the radius Y.1 of the tip 6. The needle attachment feature Y.1 may also have a maximum cross-sectional equal to or less than the radius Y.1 of the tip 6.

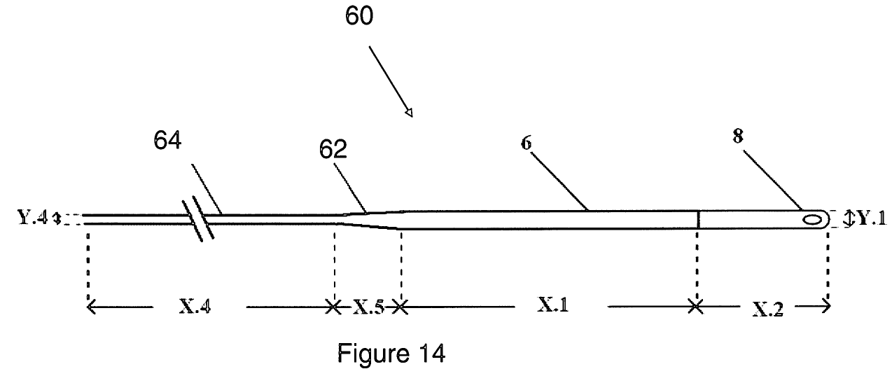

Referring generally to the Figures and particularly to FIG. 14, FIG. 14 illustrates a tapered guide wire 60 having a tapered section 62 disposed between the tip 6 and a tapering tail 64. The tapered section 62 enables the cannulated screw 16 to be advanced along the tail and toward the tip while reducing a possibility that the cannulated screw 16 might be obstructed when transitioning from the tapered tail 64 onto a position about the tip 6.

The tapered section 62 has a cross sectional radius Y.4 proximate to the tail and increases to a maximum cross-sectional radius Y.1 proximate to the tip 6. The tapered section may be comprised within either the tapered tail 64 or the tip 6 in various alternate preferred embodiments of the present invention. The tapered guide wire 60 may be a homogenous length of material, such as a length of nitinol. Alternatively, the tip 6 may be a nitinol wire section having a radius Y.1 in the range from 0.7 to 3.0 millimeters, and comprising the tapered section 62 of tapered nitinol wire, or other suitable material known in the art. The tapered tail 64 may be comprised of nitinol wire surgical suture, or other suitable material known in the art, and preferably having a maximum cross-sectional radius Y.4 in the range of 0.5 to 1.2 millimeters.

The tapered tail length X.4 is preferably in the range between 0.2 meters to 2.0 meters, and more preferably in the range between 0.3 meters and 0.6 meters.

The tapered section length X.5 may be a long as the cannulated screw length X.3, and/or preferably in the range between 5 millimeters to 20 meters, and more preferably in the range between 10 millimeters and 15 millimeters.

The foregoing disclosures and statements are illustrative only of the Present Invention, and are not intended to limit or define the scope of the Present Invention. The above description is intended to be illustrative, and not restrictive. Although the examples given include many specificities, they are intended as illustrative of only certain possible embodiments of the Present Invention. The examples given should only be interpreted as illustrations of some of the preferred embodiments of the Present Invention, and the full scope of the Present Invention should be determined by the appended claims and their legal equivalents. Those skilled in the art will appreciate that various adaptations and modifications of the just-described preferred embodiments can be configured without departing from the scope and spirit of the Present Invention. Therefore, it is to be understood that the Present Invention may be practiced other than as specifically described herein. The scope of the Present Invention as disclosed and claimed should, therefore, be determined with reference to the knowledge of one skilled in the art and in light of the disclosures presented above.

I claim:

1. A method of fixation of a tissue graft in a bone socket, a first end of the tissue graft coupled with a strand, and the bone socket having (1.) a socket for placement of a first end of the tissue graft and (2.) a bone socket aperture for removal of the strand from the bone socket, the method comprising:
a. coupling a guide wire to the strand;
b. pulling the strand through the bone socket aperture to pull the first end of the tissue graft into towards the bone socket; and
c. driving a cannulated screw along the guide wire toward the strand aperture, whereby the cannulated screw provides an interference fixation of the tissue graft within the bone socket.

2. The method of claim 1, further comprising withdrawing the strand completely from the bone socket through the bone socket aperture.

3. The method of claim 1, further comprising withdrawing the guide wire from the bone socket.

4. The method of claim 1, wherein the first end is a bone graft.

5. The method of claim 4, wherein the bone graft is coupled with a tendon tissue.

6. The method of claim 1, wherein the tissue graft is a tendon tissue, and the first end of the tissue graft is comprised of a portion of the tendon tissue folded about the strand.

7. The method of claim 1, wherein the guide wire comprises a loop and the strand extends through the loop.

8. The method of claim 7, further comprising withdrawing the strand from the bone socket through the bone socket aperture.

9. The method of claim 7, wherein the cannulated screw is configured to permit withdrawal of the guide wire loop through a guide channel of the cannulated screw.

10. The method of claim 1, wherein the guide wire comprises a hook, and the hook is configured to detachably couple the guide wire to the strand.

11. The method of claim 10, wherein the cannulated screw is configured to permit withdrawal of the guide wire hook through a guide channel of the cannulated screw.

12. The method of claim 10, wherein the guide wire further comprises an opposing hook, and the opposing hook is configured to detachably couple the guide wire to the strand.

13. The method of claim 1, wherein the bone is a human femur.

14. A surgical device for securing a tissue graft to a bone socket, the surgical device comprising:
a. a strand, the strand coupled to the tissue graft, and the strand configured for extending through a strand aperture of the bone socket; and
b. a guide wire, the guide wire coupled to the strand, whereby a cannulated screw may travel along the guide wire to form an interference fixation of the tissue graft within the bone socket.

15. The device of claim 14, wherein the guide wire comprises nitinol.

16. The device of claim 14, further comprising a cannulated screw, the cannulated screw configured to travel along the guide wire and the guide wire configured to extend through the cannulated screw.

17. The device if claim 14, wherein the guide wire further comprises a guide wire loop, the strand extending through the guide wire loop and the guide wire loop configured for removal through an inner channel of the cannulated screw.

18. The device if claim 14, wherein the guide wire further comprises a guide wire hook, the guide wire hook for detachably coupling the guide wire to the strand, and the guide wire hook configured for removal through an inner channel of the cannulated screw.

19. The device of claim 18, wherein the guide further comprises an opposing guide wire hook, the opposing guide wire hook for detachably coupling the guide wire to the strand, and the opposing guide wire hook configured for removal through an inner channel of the cannulated screw.

20. The device of claim 19, wherein the bone is socket is located in a human femur.
